(19) United States
(12) Patent Application Publication
(10) Pub. No.: US 2020/0367756 A1
(43) Pub. Date: Nov. 26, 2020

(54) METHOD AND SYSTEM FOR IN VIVO DETECTION OF ADIPOSE TISSUE BROWNING

(71) Applicant: Agency for Science, Technology and Research, Singapore (SG)

(72) Inventors: Malini Olivo, Singapore (SG); Kapil Dev, Singapore (SG); Dinish Unnimadhava Kurup Soudamini Amma, Singapore (SG); Renzhe Bi, Singapore (SG)

(21) Appl. No.: 16/960,306

(22) PCT Filed: Jan. 14, 2019
(86) PCT No.: PCT/SG2019/050020

§ 371 Date: Jul. 6, 2020

(30) Foreign Application Priority Data
Jan. 12, 2018 (SG) 10201800320S

(51) Int. Cl. A61B 5/00; A61B 5/103

(52) U.S. Cl. CPC A61B 5/0075; A61B 2503/40; A61B 2562/0233; A61B 5/1032

(57) ABSTRACT

Provided are a system and a method for in vivo detection of adipose tissue browning. The system includes a fiber probe configured to illuminate light on an adipose tissue site; a spectrometer configured to obtain diffuse reflectance spectrum information from the adipose tissue site; a chromophore measure determining module configured to determine a fraction of lipid chromophore with respect to lipid chromophore and water chromophore based on spectrally unmixing the diffuse reflectance spectrum information in a region of 1050 nm to 1400 nm; and a browning detector module configured to detect adipose tissue browning based on the fraction determined above.

### CROSS-REFERENCE TO RELATED APPLICATION

This application claims the benefit of priority of Singapore Patent Application

No. 10201800320S filed 12 Jan. 2018, the content of which being hereby incorporated by reference in its entirety for all purposes.

### TECHNICAL FIELD

The present invention generally relates to a method and system for in vivo detection of adipose tissue browning, and a method of detecting adipose tissue browning based on diffuse reflectance spectrum information.

### BACKGROUND

Adipose tissue has been recognized primarily as a lipid metabolism organ that stores excess energy in the form of triglycerides and breaks them down into fatty acids through lypolysis under hormonal stimulation. White adipose tissue (WAT) and brown adipose tissue (BAT) are anatomically and developmentally distinct fat tissues with different functions. For example, while WAT mainly stores large amount of triglycerides in lipid droplets, BAT catabolizes energy sources and generates heat primarily through the function of uncoupling reaction mediated by uncoupling protein-1 (UCP1). It is generally thought that BAT is abundant in rodents and infants but not in human adults. However, it was recently discovered that there are inducible, brown-like adipocytes (which may also interchangeably be referred to as browning, beige or brite adipocytes) dispersed inside WAT. These beige or browning adipocytes are mainly localized in subcutaneous WAT and exhibit genetic and biological characteristics of BAT reported in human adults. Its resemblance to human BAT activity and potential of browning adipocytes to systemically burn excessive calories prompted more extensive biological investigation and potential applications of these cells as a therapeutic target against obesity and obesity-associated complications, such as diabetes.

There have been attempts to establish imaging methods to detect classical BAT activities in animals and humans. There are sufficient reports on in vivo imaging and detection of classical BAT and WAT using common imaging approaches such as magnetic resonance imaging (MRI) and positron emission tomography (PET). PET with computed tomography (CT) has been by far the most commonly used protocol both in animal models and human studies by injecting ¹⁸F-fluorodeoxyglucose (FDG) as a contrast agent and by detecting the hot spots of glucose uptake in vivo. However, MRI has advantages over PET as a technique, which neither requires exogenous tracer to visualize BAT nor ionizing radiation. MRI imaging has been widely reported to investigate classical BAT and WAT by differentiating its intrinsic morphology in terms of blood perfusion, vascularization by capillaries, and so on. However, so far there does not appear to be any reports of PET/MRI imaging for quantifying browning process in vivo.

Recently, brown fat metabolism has been reported by imaging haemoglobin gradients in animals and humans utilizing multi-spectral optoacoustic imaging (MSOT) within spectral range 700 to 970 nm. The principle behind the investigation is that the BAT activation can be measured by resolving haemoglobin oxygenation gradients acting as representatives of local oxygen utilization and blood influx. In animals, ex vivo and in vivo MSOT measurement showed spectral differentiation between interscapular (IS) BAT, brown inguinal (IG) WAT (beige) and IG WAT within this wavelength range. In another recent study, the metabolic characteristics of different types of adipocytes in mice during adrenergically stimulated thermogenesis in vivo using thermal imaging have been investigated. The redox states of classical BAT and WAT in live mice have also been characterized using endogenous fluorescence approach.

Recently, detection of classical WAT and BAT in vivo using optical techniques, such as diffuse optical spectroscopy and imaging (DOSI) or time resolved spectroscopy has been reported. However, unlike BAT, browning or beige adipocytes (or beige fat) are sparsely and transiently present inside subcutaneous WAT, formed as a result of selective activation by stimuli, such as cold exposure or adrenergic agonists. In this regard, the sparse population of beige adipocytes makes its in vivo quantitative detection or imaging much more challenging than that of classical BAT, and there does not appear to be any report on the successful or effective in vivo detection of adipose tissue browning so far.

A need therefore exists to provide a method and system for in vivo detection of adipose tissue browning, and a method of detecting adipose tissue browning based on diffuse reflectance spectrum information. It is against this background that the present invention has been developed.

### SUMMARY

According to a first aspect of the present invention, there is provided a system for in vivo detection of adipose tissue browning, the system comprising:

a fiber probe configured to illuminate light on an adipose tissue site;

a spectrometer configured to obtain diffuse reflectance spectrum information based on diffuse reflected light from the adipose tissue site in response to the light illuminated thereon; and

a chromophore measure determining module configured to determine a quantitative measure of a first type of chromophore at the adipose tissue site based on spectrally unmixing the diffuse reflectance spectrum information; and

a browning detector module configured to detect adipose tissue browning at the adipose tissue site based on the quantitative measure of the first type of chromophore determined.

In various embodiments according to the first aspect, the diffuse reflectance spectrum information is spectrally unmixed based on a lookup table which, for each combination of a plurality of combinations of values of a plurality of tissue optical property parameters, maps the combination to a corresponding diffuse reflectance value.

In various embodiments according to the first aspect, the plurality of tissue optical property parameters comprises a reduced scattering coefficient and an absorption coefficient.

In various embodiments according to the first aspect, the absorption coefficient is dependent on the quantitative measure of the first type of chromophore at the adipose tissue site, and the quantitative measure of the first type of chromophore is determined based on a comparison between the diffuse reflectance spectrum information obtained and a modeled diffuse reflectance spectrum generated based on the quantitative measure of the first type of chromophore using the lookup table.

In various embodiments according to the first aspect, the absorption coefficient is dependent on quantitative measures of a plurality of types of chromophores, respectively, the plurality of types of chromophores including the first type of chromophore.

In various embodiments according to the first aspect, the quantitative measure of the first type of chromophore is a fraction of the first type of chromophore with respect to the plurality of types of chromophores.

In various embodiments according to the first aspect, the plurality of types of chromophores comprises lipid chromophore and water chromophore, and the first type of chromophore is the lipid chromophore.

In various embodiments according to the first aspect, the diffuse reflectance spectrum information is spectrally unmixed in a wavelength region of about 1050 nm to about 1400 nm.

In various embodiments according to the first aspect, the fiber probe comprises a source fiber channel and a plurality of detector fiber channels extending longitudinally within the fiber probe.

In various embodiments according to the first aspect, in a cross-section of the fiber probe, the plurality of detector fiber channels has a circular arrangement about the source fiber channel.

According to a second aspect of the present invention, there is provided a method of in vivo detection of adipose tissue browning, the method comprising:

illuminating light on an adipose tissue site using a fiber probe;

obtaining diffuse reflectance spectrum information based on diffuse reflected light from the adipose tissue site in response to the light illuminated thereon;

determining a quantitative measure of a first type of chromophore at the adipose tissue site based on spectrally unmixing the diffuse reflectance spectrum information; and

detecting adipose tissue browning at the adipose tissue site based on the quantitative measure of the first type of chromophore determined.

In various embodiments according to the second aspect, the diffuse reflectance spectrum information is spectrally unmixed based on a lookup table which, for each combination of a plurality of combinations of values of a plurality of tissue optical property parameters, maps the combination to a corresponding diffuse reflectance value.

In various embodiments according to the second aspect, the plurality of tissue optical property parameters comprises a reduced scattering coefficient and an absorption coefficient.

In various embodiments according to the second aspect, the absorption coefficient is dependent on the quantitative measure of the first type of chromophore at the adipose tissue site, and the quantitative measure of the first type of chromophore is determined based on a comparison between the diffuse reflectance spectrum information obtained and a modeled diffuse reflectance spectrum generated based on the quantitative measure of the first type of chromophore using the lookup table.

In various embodiments according to the second aspect, the absorption coefficient is dependent on quantitative measures of a plurality of types of chromophores, respectively, the plurality of types of chromophores including the first type of chromophore.

In various embodiments according to the second aspect, the quantitative measure of the first type of chromophore is a fraction of the first type of chromophore with respect to the plurality of types of chromophores.

In various embodiments according to the second aspect, the plurality of types of chromophores comprises lipid chromophore and water chromophore, and the first type of chromophore is the lipid chromophore.

In various embodiments according to the second aspect, the diffuse reflectance spectrum information is spectrally unmixed in a wavelength region of about 1050 nm to about 1400 nm.

In various embodiments according to the second aspect, the fiber probe comprises a source fiber channel and a plurality of detector fiber channels extending longitudinally within the fiber probe.

In various embodiments according to the second aspect, in a cross-section of the fiber probe, the plurality of detector fiber channels has a circular arrangement about the source fiber channel.

According to a third aspect of the present invention, there is provided a method of detecting adipose tissue browning based on diffuse reflectance spectrum information, the method comprising:

receiving diffuse reflectance spectrum information with respect to an adipose tissue site;

determining a quantitative measure of a first type of chromophore at the adipose tissue site based on spectrally unmixing the diffuse reflectance spectrum information; and

detecting adipose tissue browning at the adipose tissue site based on the quantitative measure of the first type of chromophore determined.

In various embodiments according to the third aspect, the diffuse reflectance spectrum information is spectrally unmixed based on a lookup table which, for each combination of a plurality of combinations of values of a plurality of tissue optical property parameters, maps the combination to a corresponding diffuse reflectance value.

In various embodiments according to the third aspect, the plurality of tissue optical property parameters comprises a reduced scattering coefficient and an absorption coefficient.

In various embodiments according to the third aspect, the absorption coefficient is dependent on the quantitative measure of the first type of chromophore at the adipose tissue site, and the quantitative measure of the first type of chromophore is determined based on a comparison between the diffuse reflectance spectrum information obtained and a modeled diffuse reflectance spectrum generated based on the quantitative measure of the first type of chromophore using the lookup table.

In various embodiments according to the third aspect, the absorption coefficient is dependent on quantitative measures of a plurality of types of chromophores, respectively, the plurality of types of chromophores including the first type of chromophore.

In various embodiments according to the third aspect, the quantitative measure of the first type of chromophore is a fraction of the first type of chromophore with respect to the plurality of types of chromophores.

In various embodiments according to the third aspect, the plurality of types of chromophores comprises lipid chromophore and water chromophore, and the first type of chromophore is the lipid chromophore.

In various embodiments according to the third aspect, the diffuse reflectance spectrum information is spectrally unmixed in a wavelength region of about 1050 nm to about 1400 nm.

### BRIEF DESCRIPTION OF THE DRAWINGS

Embodiments of the present invention will be better understood and readily apparent to one of ordinary skill in the art from the following written description, by way of example only, and in conjunction with the drawings, in which:

FIG. 1 depicts a schematic flow diagram of a method of in vivo detection of adipose tissue browning according to various embodiments of the present invention;

FIG. 2 depicts a schematic flow diagram of a method of detecting adipose tissue browning based on diffuse reflectance spectrum information according to various embodiments of the present invention;

FIG. 3 depicts a schematic drawing of a system for in vivo detection of adipose tissue browning according to various embodiments of the present invention;

FIG. 4 depicts a schematic block diagram of an exemplary computer system which may be used to realize or implement the computer system as depicted in FIG. 3;

FIG. 5 depicts a schematic drawing of an example setup of in vivo DRS measurement system for obtaining diffuse reflectance spectrum information (raw diffuse reflectance spectrum information) in a mouse model according to various example embodiments of the present invention;

FIG. 6A depicts a flow diagram of a method (or forward model) configured to generate a modeled diffuse reflectance spectrum using a Monte Carlo Lookup Table (MCLUT) according to various example embodiments of the present invention, for an iteration process;

FIG. 6B depicts an example MCLUT for mapping various combinations of values of reduced scattering coefficient μ′ₛ (e.g., along an x-axis) and absorption coefficient μₐ (e.g., along a y-axis) to the corresponding diffuse reflectance value (e.g., along a z-axis), according to various example embodiments of the present invention;

FIG. 6C depicts a flow diagram of a method (or inverse model or iteration process) configured to evaluate various input parameters for minimizing the degree of difference between the modeled diffuse reflectance spectrum obtained and the measured diffuse reflectance spectrum obtained, so as to estimate various parameters, including the lipid fraction, at the adipose tissue site, according to various example embodiments of the present invention;

FIG. 7 depicts a typical or representative intensity normalized diffuse reflectance spectrum measured for BAT, WAT from the control mice and beige adipose tissue from the CL injected mice within 1050 to 1350 nm wavelength range, according to various example embodiments of the present invention;

FIGS. 8A and 8B illustrate comparison of lipid fraction (S_{LW}) calculated for BAT, beige (treated WAT) and control WAT (FIG. 8A) and for WAT at inguinal (IG WAT), perirenal (PR WAT), perigonadal (PG WAT) and interscapular (IS WAT) locations in control mice, according to various example embodiments of the present invention;

FIGS. 9A and 9B illustrate independent two-sample t test between control WAT and BAT (P value=0.003) (FIG. 9A) and control WAT and beige (treated WAT) (P value=0.013) (FIG. 9B), which signify clear difference of mean value for related sample populations, according to various example embodiments of the present invention;

FIG. 10A illustrates a relative mRNA expression of Ucp1 in BAT (C), BAT (CL), WAT and beige. The mRNA expression level of Ucp1 is normalized relative to Gapdh mRNA expression, according to various example embodiments of the present invention;

FIG. 10B depicts a western blot analysis of UCP1 and loading control β-ACTIN, according to various example embodiments of the present invention. UCP1 shows higher expression upon CL administration in WAT (beige);

FIGS. 11A and 11B show hematoxylin and eosin (H&E) staining images of WAT treated with either saline (WAT) or CL (beige) (FIG. 11A) and BAT upon saline BAT (C) or CL BAT (CL) treatments (FIG. 11B), according to various example embodiments of the present invention. Beige showed multilocular adipocytes as a result of browning. Scale bar represents 100 μm (representative images from n=4 in each group); and

FIGS. 12A and 12B show CL treatment induced UCP1 expression in CL treated WAT (beige). FIG. 12A show UCP1 IHC images of WAT either treated with saline (WAT) or CL (beige), and FIG. 12B show BAT upon saline BAT (C) or CL BAT (CL) treatments, according to various example embodiments of the present invention. Beige showed increased UCP1 staining as pointed by arrows as compared to WAT. Scale bar represents 100 μm (representative images from n=4 in each group).

### DETAILED DESCRIPTION

White adipose tissue (WAT) and brown adipose tissue (BAT), biologically function in an opposite way in energy metabolism. BAT induces energy consumption by heat production while WAT mainly stores energy in the form of triglycerides. Recent progress in the conversion of WAT cells to brown-like adipocytes (which may also interchangeably be referred to as browning, beige or brite adipocytes) in animals, having functional similarity to BAT, spurred a great interest in developing the next generation therapeutics in the field of metabolic disorders.

According to various embodiments of the present invention, optical spectroscopy techniques have the unique advantage to serve as a promising tissue analysis tool because it provides accurate quantitative information based on their difference in light absorption and scattering properties across visible-near infra-red (NIR) range. For example, broadband light incident on tissue surface follows certain paths depending on the absorption coefficient (μₐ) (e.g., which defines how far the light travels through the medium before getting absorbed) and the reduced scattering coefficient (μ′ₛ) (e.g., which determines how far the light travel in the medium before being scattered). The absorption of light by tissues in the visible-NIR region may primarily be due to the endogenous chromophores such as hemoglobin, melanin, lipid, water, and so on; whereas, the scattering within the tissue may be attributed to local change in refractive index. Both parameters are highly wavelength dependent. DRS has been used for a number of biomedical applications including epithelial tissue characterization, brain study, breast cancer detection and surgical margin assessment, skin pigmentation research and so on.

In general, DRS, as the name suggests, measures the reflected light from the diffused specimen under interrogation. Depending upon the sample, the diffuse reflected light has the signature of its absorption coefficient, colour, texture, roughness and so on. In Biophotonics, the reflected white light in visible-NIR region may be affected by tissue endogenous chromophores such as hemoglobin, melanin, lipid, water and so on.

Recently, detection of classical WAT and BAT in vivo using optical techniques, such as diffuse optical spectroscopy and imaging (DOSI) or time resolved spectroscopy, has been reported. For example, in a study, DOSI was used to detect the changes in subcutaneous adipose tissue during weight loss in humans. The perfusion status and water fraction in the 650 to 1000 nm window were used for the characterization. For example, in another study, NIR-time resolved spectroscopy was used to detect the density of BAT measuring total hemoglobin and realized its characterization in humans. They successfully reported the changes in BAT density during or after long-term intervention and these results were consistent with PET-CT studies.

Similar to classical BAT, beige fat express brown-specific Ucp1 gene, and also possess high mitochondrial content and multilocular lipid droplets. However, unlike BAT, beige fat is sparsely and transiently present inside subcutaneous WAT, formed as a result of selective activation by stimuli such as cold exposure or adrenergic agonists. The sparse population of beige adipocytes makes its in vivo quantitative detection or imaging much more challenging than that of classical BAT and there does not appear to be any report on the successful or effective in vivo detection of adipose tissue browning so far.

Various embodiments of the present invention provide a method for in vivo detection of adipose tissue browning (in a subject, such as an animal or a human), and a system thereof, and in particular, based on diffuse reflectance spectroscopy (DRS).

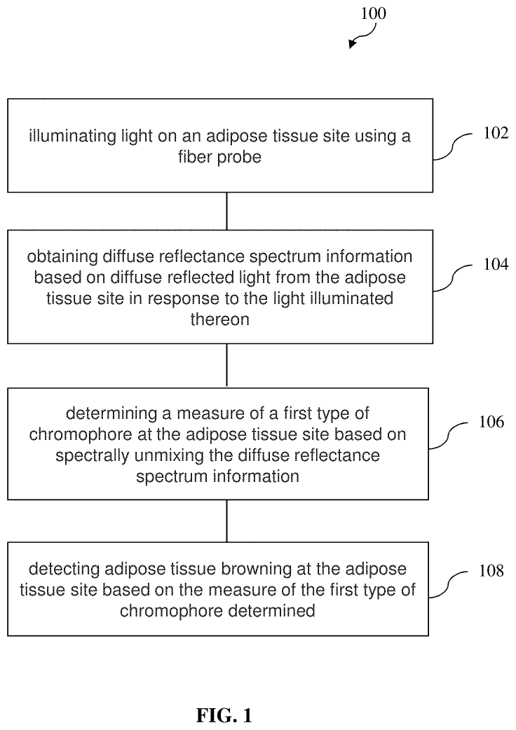

FIG. 1 depicts a schematic flow diagram of a method 100 of in vivo detection of adipose tissue browning according to various embodiments of the present invention. The method 100 comprises illuminating (at 102) light on an adipose tissue site (or adipose tissue location) using a fiber probe; obtaining (at 104) diffuse reflectance spectrum information based on diffuse reflected light from the adipose tissue site in response to the light illuminated thereon; determining (at 106) a quantitative measure of a first type of chromophore at the adipose tissue site based on spectrally unmixing the diffuse reflectance spectrum information; and detecting (at 108) adipose tissue browning at the adipose tissue site based on the quantitative measure of the first type of chromophore determined.

In various embodiments, in relation to 102, the light for illuminating the adipose tissue site (e.g., desired or target adipose tissue site) is preferably a broadband light, and more preferably, a broadband white light, from a light source (e.g., a halogen light source or lamp). In this regard, a first end (or light receiving end) of the fiber probe may be coupled (directly or indirectly) to the light source for receiving the light for illuminating the adipose tissue site and a second end (or light emitting end) of the fiber probe may be provided or positioned (e.g., handheld and movable by an operator or a user) at the adipose tissue site (e.g., through an incised skin optical window formed on a subject) such that the light emitted from the second end is able to illuminate (e.g., directly) the adipose tissue site. In various embodiments, the fiber probe may be an excitation-collection fiber with source-detector fiber separations configured to achieve a desired depth of interrogation.

In relation to 104, diffuse reflectance spectrum information may be obtained (e.g., generated) from diffuse reflected light received by a spectrometer in manner known in the art and thus need not be described herein for clarity and conciseness.

In various embodiments, in relation to 106, a quantitative measure of a chromophore may refer to any quantitative information relating to the chromophore determined or derived, such as but not limited to, an amount (e.g., concentration) of the chromophore or a proportion (or fraction or ratio) of the chromophore at a desired or target tissue site. In various embodiments, the diffuse reflectance spectrum information may be spectrally unmixed for or with respect to one or a plurality of chromophores. For example, the diffuse reflectance spectrum information obtained using a fiber probe at an adipose tissue site has mixed contributions from different chromophores existing or present in the adipose tissue site, such as haemoglobin, de-oxy haemoglobin, water, lipid and so on. In this regard, various embodiments separate or unmix the contribution with respect to one or a plurality of chromophores from the mixed diffuse reflectance spectrum by taking one or more tissue optical property parameters (e.g., absorption coefficient and reduced scattering coefficient) into consideration.

In various embodiments, in relation to 108, detecting adipose tissue browning may include a quantitative detection of the adipose tissue browning, such as the amount (e.g., concentration) or the proportion (or fraction or ratio) of browning adipocytes present at the adipose tissue site.

Accordingly, various embodiments of the present invention advantageously provide a method of in vivo detection of adipose tissue browning, which is label-free and effective.

In various embodiments, the diffuse reflectance spectrum information is spectrally unmixed based on a lookup table (LUT) which, for each combination of a plurality of combinations of values of a plurality of tissue optical property parameters, maps the combination to a corresponding diffuse reflectance value. In various embodiments, the LUT may be a Monte Carlo LUT (MCLUT), whereby each mapping (a diffuse reflectance value for a given combination of tissue optical property parameters) is determined based on a corresponding Monte Carlo simulation. In various embodiments, diffuse reflectance values for a set of combinations of the plurality of tissue optical property parameters may be computed, such as within a predetermined range of each tissue optical property parameter, as desired or as appropriate. Accordingly, the LUT establishes or provides a relationship between the diffuse reflectance value and a particular combination of a plurality of tissue optical property parameters.

In various embodiments, the plurality of tissue optical property parameters comprises a reduced scattering coefficient and an absorption coefficient.

In various embodiments, the absorption coefficient is dependent on the quantitative measure of the first type of chromophore at the adipose tissue site, and the quantitative measure of the first type of chromophore is determined based on a comparison between the diffuse reflectance spectrum information obtained and a modeled diffuse reflectance spectrum generated based on the quantitative measure of the first type of chromophore using the lookup table. In various embodiments, the dependency may be defined or expressed as a function or equation, with the quantitative measure being a parameter or variable of the function or equation. In various embodiments, the comparison may be configured to determine a degree of similarity or difference between the diffuse reflectance spectrum information obtained and the modeled diffuse reflectance spectrum generated, such as being a part of an iteration process for minimizing the degree of difference (e.g., least square difference) therebetween (with variable parameters including the quantitative measure of the first type of chromophore).

In various embodiments, the absorption coefficient is dependent on quantitative measures of a plurality of types of chromophores, respectively, at the adipose tissue site, the plurality of types of chromophores including the first type of chromophore. Similarly, the quantitative measures of the plurality of types of chromophores may be determined based on a comparison between the diffuse reflectance spectrum information obtained and a modeled diffuse reflectance spectrum generated based on the quantitative measures of the plurality of chromophores using the lookup table. Similarly, the dependency may be defined or expressed as a function or equation, with the quantitative measures being parameters or variables of the function or equation. Similarly, the comparison may be configured to determine a degree of similarity or difference between the diffuse reflectance spectrum information obtained and the modeled diffuse reflectance spectrum generated, such as being a part of an iteration process for minimizing the degree of difference (e.g., least square difference) therebetween (with variable parameters including the quantitative measures of the plurality of types of chromophores).

In various embodiments, the quantitative measure of the first type of chromophore is a fraction (or ratio or proportion) of the first type of chromophore with respect to the plurality of types of chromophores. In various embodiments, the fraction may be determined as a ratio of a concentration of the first type of chromophore to the total or combined concentration of the plurality of types of chromophores.

In various embodiments, the plurality of types of chromophores comprises lipid chromophore and water chromophore, and the first type of chromophore is the lipid chromophore. In various embodiments, the plurality of types of chromophores consist of lipid chromophore and water chromophore, that is, only lipid chromophore and water chromophore, and the first type of chromophore is the lipid chromophore. In other words, for example, the detection of adipose tissue browning according to various embodiments is advantageously only based on lipid chromophore and water chromophore at the adipose tissue site.

In various embodiments, the diffuse reflectance spectrum information is spectrally unmixed in a wavelength region of about 1050 nm to about 1400 nm. In various embodiments, the wavelength region may be from 1050 nm to 1350 nm, or from 1050 nm to about 1300 nm.

In various embodiments, the fiber probe comprises a source fiber channel and a plurality of detector fiber channels extending longitudinally within the fiber probe.

In various embodiments, in a cross-section of the fiber probe, the plurality of detector fiber channels has a circular arrangement about the source fiber channel. In other others, the plurality of detector fiber channels may be arranged around the source fiber channel in a circular manner, with the source fiber channel being at the center. In various embodiments, the plurality of detector fiber channels may be arranged to form a plurality of concentric circles with the source fiber channel being at the center.

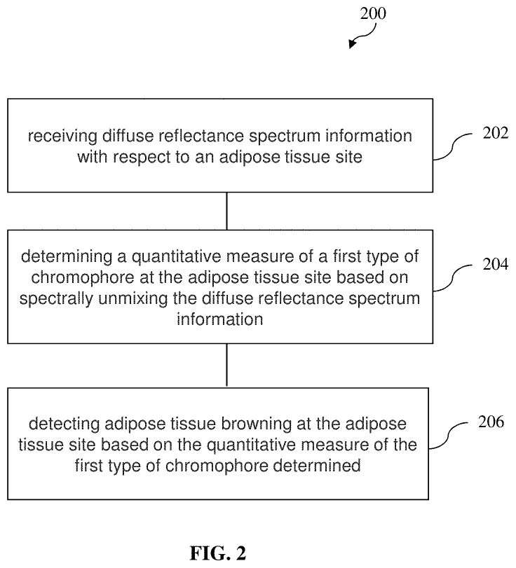

FIG. 2 depicts a schematic flow diagram of a method 200 of detecting adipose tissue browning based on diffuse reflectance spectrum information (or a method of processing diffuse reflectance spectrum information for detecting adipose tissue browning) according to various embodiments of the present invention. The method 200 comprises receiving, at 202, diffuse reflectance spectrum information with respect to an adipose tissue site; determining, at 204, a quantitative measure of a first type of chromophore at the adipose tissue site based on spectrally unmixing the diffuse reflectance spectrum information; and detecting, at 206, adipose tissue browning at the adipose tissue site based on the quantitative measure of the first type of chromophore determined.

In various embodiments, in relation to 202, the diffuse reflectance spectrum information may be the diffuse reflectance spectrum information obtained based on diffuse reflected light from an adipose tissue site in response to light illuminated thereon, as described hereinbefore with reference to 104 of FIG. 1. The above-mentioned determining, at 204, a quantitative measure and detecting, at 206, adipose tissue browning correspond to (e.g., are the same as) 106 and 108 described hereinbefore with reference to FIG. 1, the associated features according to various embodiments need not be repeated with respect to the method 200 for clarity and conciseness.

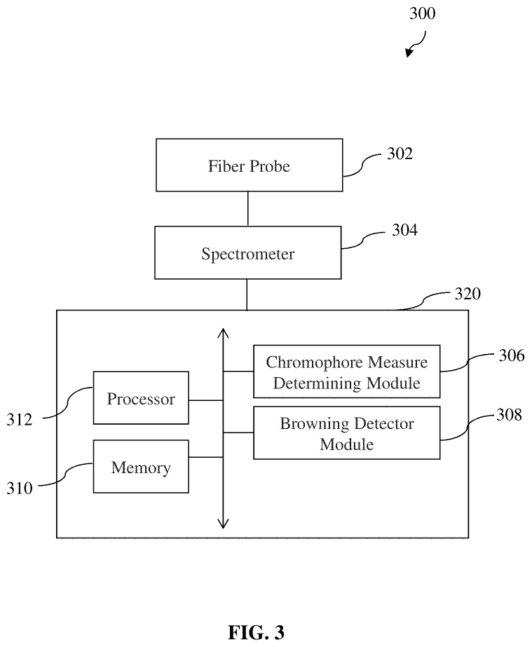

FIG. 3 depicts a schematic drawing of a system 300 for in vivo detection of adipose tissue browning according to various embodiments of the present invention, such as corresponding to the method 100 of in vivo detection of adipose tissue browning as described hereinbefore according to various embodiments of the present invention. The system 300 comprises a fiber probe 302 configured to illuminate light on an adipose tissue site; a spectrometer 304 configured to obtain diffuse reflectance spectrum information based on diffuse reflected light from the adipose tissue site in response to the light illuminated thereon; a chromophore measure determining module (or chromophore measure determining circuit) 306 configured to determine a quantitative measure of a first type of chromophore at the adipose tissue site based on spectrally unmixing the diffuse reflectance spectrum information; and a browning detector module (or browning detector circuit) 308 configured to detect adipose tissue browning at the adipose tissue site based on the quantitative measure of the first type of chromophore determined.

In various embodiments, the fiber probe 302 (e.g., hand-held fiber probe) may be communicatively coupled to the spectrometer 304 such that the diffuse reflected light collected by the fiber probe 302 may be received by the spectrometer 304 via the fiber probe 302.

It will be appreciated by a person skilled in the art that the system 300 may comprise a memory 310 and at least one processor 312 communicatively coupled to the memory 310 and configured to perform various functions/operations as described hereinbefore according to various embodiments. For example, the at least one processor 312 may be configured to determine a quantitative measure of a first type of chromophore at the adipose tissue site based on spectrally unmixing the diffuse reflectance spectrum information (e.g., corresponding to the above-mentioned chromophore measure determining module 306), and may be configured to detect adipose tissue browning at the adipose tissue site based on the quantitative measure of the first type of chromophore determined (e.g., corresponding to the above-mentioned browning detector module 308). It will also be appreciated by a person skilled in the art that the spectrometer 304 may also comprise a memory (not shown) and at least one processor (not shown) communicatively coupled to the memory and configured to perform various functions/operations of the spectrometer 304. For example, the at least one processor of the spectrometer 304 may be configured to obtain diffuse reflectance spectrum information based on diffuse reflected light from the adipose tissue site received by the spectrometer 304 in manner known in the art and thus need not be described herein for clarity and conciseness.

In various embodiments, a computing system or device 320 including the above-mentioned memory 310 and at least processor 312 may be provided and communicatively coupled (e.g., via wired or wireless communications) to the spectrometer 304 for receiving the diffuse reflectance spectrum information therefrom. In various other embodiments, the computing system or device 320 may be integrated with the spectrometer 304 such that the at least one processor of the spectrometer 304 is further configured to perform the above-mentioned functions or operations of the at least one processor 312. In other words, it is not necessary that a separate computing system or device 320 be provided, and the processing of the diffuse reflectance spectrum information according to various embodiments of the present invention may be configured according to various embodiments of the present invention to be performed by the at least one processor of the spectrometer 304 for detecting adipose tissue browning at the desired or target adipose tissue site.

It will be appreciated by a person skilled in the art that a processor may be configured to perform the required functions or operations through set(s) of instructions (e.g., software modules) executable by the processor to perform the required functions or operations, such as to realize the above-mentioned chromophore measure determining module 306 and/or the browning detector module 308.

In various embodiments, the system 300 corresponds to the method 100 as described hereinbefore with reference to FIG. 1, and therefore, various functions or operations configured to be performed by the system 300 may correspond to various steps of the method 100 described hereinbefore according to various embodiments, and thus need not be repeated with respect to the system 300 for clarity and conciseness. In other words, various embodiments described herein in context of the method 100 are analogously valid for the corresponding system 300, and vice versa.

In various embodiments, there is provided a system for detecting adipose tissue browning based on diffuse reflectance spectrum information according to various embodiments of the present invention, corresponding to the method 200 of detecting adipose tissue browning based on diffuse reflectance spectrum information as described hereinbefore with reference to FIG. 2 according to various embodiments of the present invention. In various embodiments, the above-mentioned system corresponds to (e.g., is the same as) the computing system or device 320 as described hereinbefore with reference to FIG. 3, and therefore various functions or operations configured to be performed by the above-mentioned system may correspond to those to be performed by the computing system 320 as described hereinbefore, and thus need not be repeated with respect to the above-mentioned system for clarity and conciseness.

A computing system, a controller, a microcontroller or any other system providing a processing capability may be provided according to various embodiments in the present disclosure. Such a system may be taken to include one or more processors and one or more computer-readable storage mediums. For example, the system 300 described hereinbefore may include a processor (or controller) and a computer-readable storage medium (or memory) which are for example used in various processing carried out therein as described herein. A memory or computer-readable storage medium used in various embodiments may be a volatile memory, for example a DRAM (Dynamic Random Access Memory) or a non-volatile memory, for example a PROM (Programmable Read Only Memory), an EPROM (Erasable PROM), EEPROM (Electrically Erasable PROM), or a flash memory, e.g., a floating gate memory, a charge trapping memory, an MRAM (Magnetoresistive Random Access Memory) or a PCRAM (Phase Change Random Access Memory).

In various embodiments, a “circuit” may be understood as any kind of a logic implementing entity, which may be special purpose circuitry or a processor executing software stored in a memory, firmware, or any combination thereof. Thus, in an embodiment, a “circuit” may be a hard-wired logic circuit or a programmable logic circuit such as a programmable processor, e.g., a microprocessor (e.g., a Complex Instruction Set Computer (CISC) processor or a Reduced Instruction Set Computer (RISC) processor). A “circuit” may also be a processor executing software, e.g., any kind of computer program, e.g., a computer program using a virtual machine code, e.g., Java. Any other kind of implementation of the respective functions which will be described in more detail below may also be understood as a “circuit” in accordance with various alternative embodiments. Similarly, a “module” may be a portion of a system according to various embodiments in the present invention and may encompass a “circuit” as above, or may be understood to be any kind of a logic-implementing entity therefrom.

Some portions of the present disclosure are explicitly or implicitly presented in terms of algorithms and functional or symbolic representations of operations on data within a computer memory. These algorithmic descriptions and functional or symbolic representations are the means used by those skilled in the data processing arts to convey most effectively the substance of their work to others skilled in the art. An algorithm is here, and generally, conceived to be a self-consistent sequence of steps leading to a desired result. The steps are those requiring physical manipulations of physical quantities, such as electrical, magnetic or optical signals capable of being stored, transferred, combined, compared, and otherwise manipulated.

In addition, the present specification also at least implicitly discloses a computer program or software/functional module, in that it would be apparent to the person skilled in the art that various steps of the methods described herein (e.g., 104, 106, 108, 202, 204 and 206) may be put into effect by computer code. The computer program is not intended to be limited to any particular programming language and implementation thereof. It will be appreciated that a variety of programming languages and coding thereof may be used to implement the teachings of the disclosure contained herein. Moreover, the computer program is not intended to be limited to any particular control flow. There are many other variants of the computer program, which can use different control flows without departing from the spirit or scope of the invention. It will be appreciated by a person skilled in the art that various modules described herein (e.g., chromophore measure determining module 306 and the browning detector module 308) may be software module(s) realized by computer program(s) or set(s) of instructions executable by a computer processor to perform the required functions, or may be hardware module(s) being functional hardware unit(s) designed to perform the required functions. It will also be appreciated that a combination of hardware and software modules may be implemented.

Furthermore, one or more of the steps of a computer program/module or method described herein may be performed in parallel rather than sequentially.

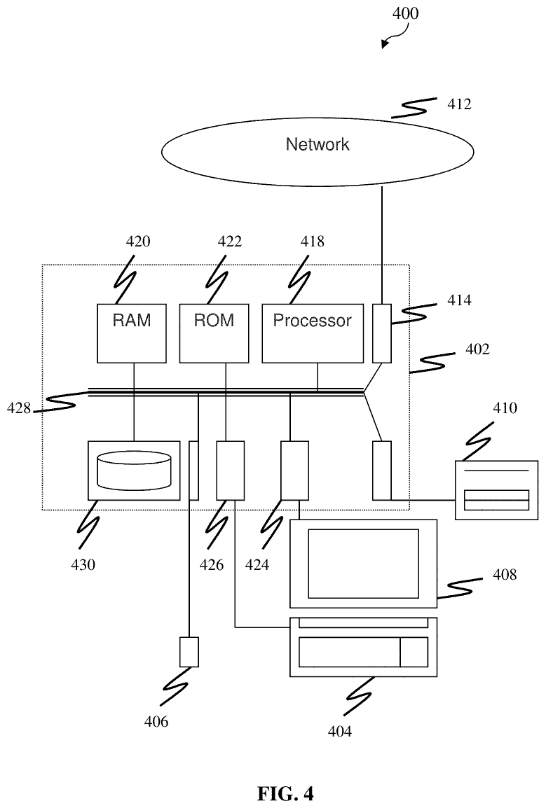

In various embodiments, the above-mentioned computer system (or computing system) 320 may be realized by any computer system (e.g., portable or desktop computing system), such as a computer system 400 as schematically shown in FIG. 4 as an example only and without limitation. Various methods/operations or functional modules (e.g., the chromophore measure determining module 306 and/or the browning detector module 308) may be implemented as software, such as a computer program being executed within the computer system 400, and instructing the computer system 400 (in particular, one or more processors therein) to conduct the methods/functions of various embodiments described herein. The computer system 400 may comprise a computer module 402, input modules, such as a keyboard 404 and a mouse 406, and a plurality of output devices such as a display 408, and a printer 410. The computer module 402 may be connected to a computer network 412 via a suitable transceiver device 414, to enable access to e.g. the Internet or other network systems such as Local Area Network (LAN) or Wide Area Network (WAN). The computer module 402 in the example may include a processor 418 for executing various instructions, a Random Access Memory (RAM) 420 and a Read Only Memory (ROM) 422. The computer module 402 may also include a number of Input/Output (I/O) interfaces, for example I/O interface 424 to the display 408, and I/O interface 426 to the keyboard 404. The components of the computer module 402 typically communicate via an interconnected bus 428 and in a manner known to the person skilled in the relevant art.

It will be appreciated by a person skilled in the art that the terminology used herein is for the purpose of describing various embodiments only and is not intended to be limiting of the present invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

In order that the present invention may be readily understood and put into practical effect, various example embodiments of the present invention will be described hereinafter by way of examples only and not limitations. It will be appreciated by a person skilled in the art that the present invention may, however, be embodied in various different forms or configurations and should not be construed as limited to the example embodiments set forth hereinafter. Rather, these example embodiments are provided so that this disclosure will be thorough and complete, and will fully convey the scope of the present invention to those skilled in the art.

Various example embodiments of the present invention provides a quantitative in vivo detection of adipose tissue browning using diffuse reflectance spectroscopy (DRS). In particular, various example embodiments provide a label-free quantitative in vivo detection (e.g., including monitoring) of adipose tissue browning process using DRS in the NIR II optical window, and more particularly, in the wavelength region of about 1050 nm to about 1400 nm within the NIR II optical window (about 1000 nm to about 1700 nm), which has been found to be advantageous for detecting (e.g., quantitative analysis of) adipose tissue browning according to various example embodiments of the present invention.

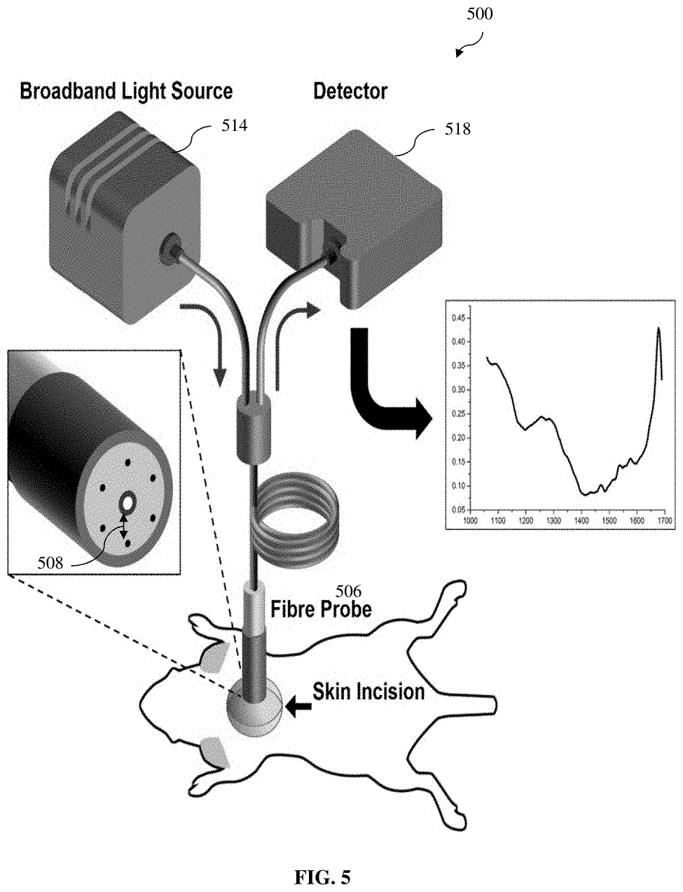

In particular, for illustration purpose only and to demonstrate the effectiveness of the method of detecting adipose tissue browning in vivo using DRS, the method and associated experimental results or observations according to various example embodiments will now be described based on a mouse model as shown in FIG. 5. In particular, FIG. 5 depicts a schematic drawing of an example setup of in vivo DRS measurement system 500 for obtaining (e.g., generating or measuring) diffuse reflectance spectrum information (raw diffuse reflectance spectrum information) in a mouse model with partial skin incision using DRS spectroscopy in the NIR II optical window. In this regard, browning was induced in mice for 7 days by β-adrenergic administration followed by DRS measurement in the 1050 to 1350 nm range using an excitation-collection fiber probe 506 with source-detector fiber separations 508 configured to achieve a desired or sufficient depth of interrogation. The measured diffuse reflectance spectrum information obtained at the adipose tissue site was then processed according to various example embodiments to extract or derive water and lipid chromophore fractions (or water and lipid proportions or ratios) at the adipose tissue site in the 1050 nm to 1350 nm wavelength range. The water and lipid chromophore fractions were estimated from the measured DRS spectra by employing a lookup table (LUT) based on inverse Monte-Carlo modelling. In other words, the measured DRS spectra at the adipose tissue site is spectrally unmixed particularly for water and lipid chromophores using the Monte-Carlo based LUT within the wavelength range of 1050 nm to 1400 nm, or more preferably, 1050 nm to 1350 nm. In the experimental data obtained, the estimated lipid fraction value showed a substantially linear decrease (gradual decrease) from WAT to BAT with an intermediate value for beige tissue. This measured change is in line with the expected decrease in lipid levels in BAT compared to WAT previously reported in the art using MRI studies. Accordingly, the browning of WAT in a mouse model was successfully quantified by estimating the lipid fraction, which serves as an endogenous marker. The in vivo browning process was also confirmed with standard molecular and biochemical assays.

The example setup 400 and various associated components or materials and methods or processes according to various example embodiments will now be described further detail below.

### Animals

Young 6 to 8 weeks old BALB/c nude mice obtained from In Vivos, Singapore were given free access to chow diet and water. The animal experimental procedures were all performed in accordance with guidelines and approval by the Institutional Animal Care and Use Committee (IACUC) of Biological Resource Centre (BRC) Singapore.

### Induction of Browning In Vivo in Mice

Mice (n=4; each group) were injected daily with either CL 316, 243 hydrate (1 mg/kg body weight intraperitoneally in test group (CL) or equal volume of saline in control (C) group animals for 7 days. After the in vivo DRS experiment, the mice was euthanized and fresh WAT tissues in control (C) and CL group were extracted and immediately frozen for RNA and protein expression analyses. WAT from the inguinal region and BAT from the interscapular region were stored in 10% normal buffered formalin (NBF) for hematoxylin and eosin (H&E) staining and Immunohistochemistry (IHC). BAT tissues extracted from mice injected with saline is marked as C BAT while BAT from CL injected mice as CL BAT. Similarly, WAT and beige refer to the WATs in saline (control) and CL injected group, respectively.

### DRS Instrumentation

A customized fiber optic probe according to various example embodiments of the present invention was used to illuminate different adipose tissue sites using a broadband white light source and to acquire the diffuse reflectance spectrum. The diffuse reflectance spectrum was measured in the NIR range of 1000 to 1700 nm for studying the water and lipid concentration changes due to browning of adipose tissue. The illumination fiber present at the center of the fiber optic probe 506 was connected to Avalight-HAL broadband tungsten halogen white light 514 source providing wavelengths spanning from 360 nm to 2500 nm with an integrated shutter. As shown in FIG. 5 according to various example embodiments, at the distal end (e.g., corresponding to the “first end” described hereinbefore) of the fiber probe 508, the single illumination fiber is surrounded with six collection fibers at an equal separation 508 from center of 2.7 mm on a circular periphery (e.g., spaced apart along the circular periphery equally) and are coupled together to connect it to the thermo-electric cooled Avantes NIR spectrometer (AvaSpec-NIR512-1.7TEC) 518 having 400 L/mm grating blazed at 1600 nm. The source detector separation (SDS) in the fiber optic probe 506 is 2.7 mm. In various example embodiments, the illumination fiber and collection fibers are multi-mode fibers with the core diameter size of 600 μm and 200 μm, respectively.

In various example embodiments, the light source may be a laser light source configured to provide super continuum laser, and the SDS in the fiber probe may be configured for penetrating deep into tissue for real-time in vivo adipose tissue browning (or fat browning) measurement.

### In Vivo DRS Measurement

As mentionen hereinbefore, FIG. 5 depicts a schematic drawing of in vivo DRS measurement system to acquire raw diffuse reflectance spectra for various adipose tissue according to various example embodiments of the present invention. The white light source 514 connected with the spectrometer 518 were allowed to warm up for at least 15 minutes prior to the DRS measurement. The room light was switched off in order to minimize the background light and the background spectrum B(λᵢ) was recorded by the spectrometer 518 after switching off the white light source input. In various example embodiments, the Ocean optics WS-1-SL spectrally flat reflectance standard was used to capture a reference spectrum for further normalization of the raw diffuse reflectance spectrum measurements from the adipose tissue.

Animals were kept under continuous isoflurane anesthesia and a small incision on skin was made to create an optical window at various fat depots (interscapular (IS), inguinal (IG), perirenal (PR) and perigonadal (PG)) to allow the fiber probe 506 to be positioned close to the tissue. The integration time used to acquire the reference spectrum or any other adipose tissue raw diffuse reflectance spectrum was 0.2 seconds. The fiber probe 506 was placed perpendicular to the interrogation area (desired or target adipose tissue site) through the incised skin optical window. To minimize the measurement errors, 10 raw spectra were captured at different native locations from different depots of fats.

### Spectral Processing

All raw adipose tissue diffuse reflectance spectra S(λᵢ) and diffuse reflectance calibration standard spectra C(λᵢ) were background corrected by subtracting the background spectrum B(λᵢ). The intensity normalized diffuse reflectance spectrum R(λᵢ) for each recording (measured diffuse reflectance spectrum information) was then calculated by Equation (1) below,

$$R ( {λ}_{i} ) = \frac{S ( {λ}_{i} ) - B ( {λ}_{i} )}{C ( {λ}_{i} ) - B ( {λ}_{i} )} \qquad ( Equation 1 )$$

The intensity normalized diffuse reflectance spectrum R(λᵢ) for different adipose tissue was further processed in the specific NIR II region of 1050 to 1350 nm wavelength range to spectrally unmix for water and lipid chromophores using Monte Carlo Lookup table (MCLUT) spectral unmixing, as will be described in further detail below. For example, the raw adipose tissue diffuse reflectance spectra or the intensity normalized diffuse reflectance spectrum (diffuse reflectance spectrum information) may be spectrally unmixed for or with respect to one or a plurality of chromophores, and more particularly in various example embodiments, with respect to water and lipid chromophores. For example, the diffuse reflectance spectrum information obtained using a fiber probe at an adipose tissue site has mixed contributions from different chromophores existing or present in the adipose tissue site, and various embodiments separate or unmix the contribution with respect to the water and lipid chromophores from the mixed diffuse reflectance spectrum by taking one or more tissue optical property parameters (preferably, absorption coefficient and reduced scattering coefficient according to various example embodiments) into consideration.

### Analysis and Modelling

According to various example embodiments, DRS utilizes tissue absorption and scattering model in order to investigate tissue chromophore composition, and the measured diffuse reflectance spectrum is modelled and analyzed by LUT based on Monte Carlo simulations of light propagation in tissue. In other words, the MCLUT is configured to model the diffuse reflectance spectrum depending on or based on different combinations of values of the reduced scattering coefficient and absorption coefficient. The respective contribution of different chromophores with respect to their concentrations in the adipose tissue site may then be determined or estimated by spectrally unmixing the measured diffuse reflectance spectrum (diffuse reflectance spectrum information) using the MCLUT. This model has no limitations on the absorption and reduced scattering coefficients of the tissue that are used to create it. The MCLUT is created with values of reflectance from different combinations of reduced scattering and absorption coefficients.

By way of an example and without limitation, in the formation of an example MCLUT, the absorption coefficient μₐ was varied between 0.0821 and 81.45 cm⁻¹, the reduced scattering coefficient μ′ₛ was varied between 1 and 60 cm⁻¹ and the scattering anisotropy factor (g) used was 0.85. In various example embodiments, for estimating the volume fraction (i.e., total volume fraction) of a plurality of types of chromophores (e.g., lipid and water chromophore in various example embodiments, corresponding to “ƒ_{LW}” in Equation 3 below) and the respective fractions of the plurality of types of chromophores (e.g., corresponding to lipid fraction (S_{LW}) and water fraction (1−S_{LW}) in Equation 3 below), the volume fraction of the plurality of types of chromophores and the respective fractions of the plurality of types of chromophores are iterated until the least square difference is minimized between measured raw reflectance spectrum and modeled reflectance spectrum from the MCLUT. The MCLUT-based inverse model for extracting chromophore properties is known in the art, such as in R. Hennessy, S. L. Lim, M. K. Markey and J. W. Tunnell, J Biomed Opt. 18, 037003 (2013) and N. Reistad, J. Nilsson, O. V. Timmermand, C. Sturesson and S. Andersson-Engels, Proc. SPIE 9531, 9314E (2015), the contents of which are being hereby incorporated by reference in their entirety for all purposes, and thus need not be described herein for clarity and conciseness.

An exemplary method of spectrally unmixing the measured diffuse reflectance spectrum will now be described in further details. The measured diffuse reflectance spectrum is spectrally unmixed in the specific NIR II region for volume fraction of lipid and water chromophores (ƒ_{LW}) and lipid fraction (S_{LW}) (i.e., being parameters or variables of Equation 3 below in an iteration process) using MCLUT. In the example, the four parameters used in the iteration process are reduced scattering coefficient at reference wavelength (μ′ₛ(λ₀)), scattering power coefficient (b), volume fraction of lipid and water together (ƒ_{LW}) and lipid fraction (S_{LW}) to the total lipid and water concentration are defined in Equations (2) to (4) below, along with the reduced scattering coefficient μ′ₛ and absorption coefficient μₐ.

$$\begin{matrix}{μ}_{s}^{′} ( λ ) = {μ}_{s}^{′} ( {λ}_{0} ) ( \frac{λ}{{λ}_{0}} ) & ( Equation 2 ) \\ {μ}_{a} = {f}_{LW} ( {μ}_{Lipid} {S}_{LW} + {μ}_{Water} ( 1 - {S}_{LW} ) ) & ( Equation 3 ) \\ {S}_{LW} = \frac{[ Lipid ]}{[ Lipid ] + [ Water ]} & ( Equation 4 )\end{matrix}$$

In an example embodiment, b is scattering power coefficient, λ₀ is the reference wavelength=1200 nm. In addition, μ_{Lipid} and μ_{water} represent the absorption coefficient for lipid and water, respectively. Furthermore, in Equation 4, [Lipid] denotes the concentration of lipid and [Water] denotes the concentration of water at the point of interrogation (the adipose tissue site). The total volume fraction of water and lipid (ƒ_{LW}) at an adipose tissue site may be defined as, by volume, the amount of lipid and water chromophores present at the adipose tissue site as a ratio with respect to the amount of all chromophores present (e.g., oxy-haemoglobin, de-oxy haemoglobin, lipid, water and melanin) at the adipose tissue site under interrogation. In other words, out of all the chromophores present, ƒ_{LW} denotes the volume fraction of lipid and water together.

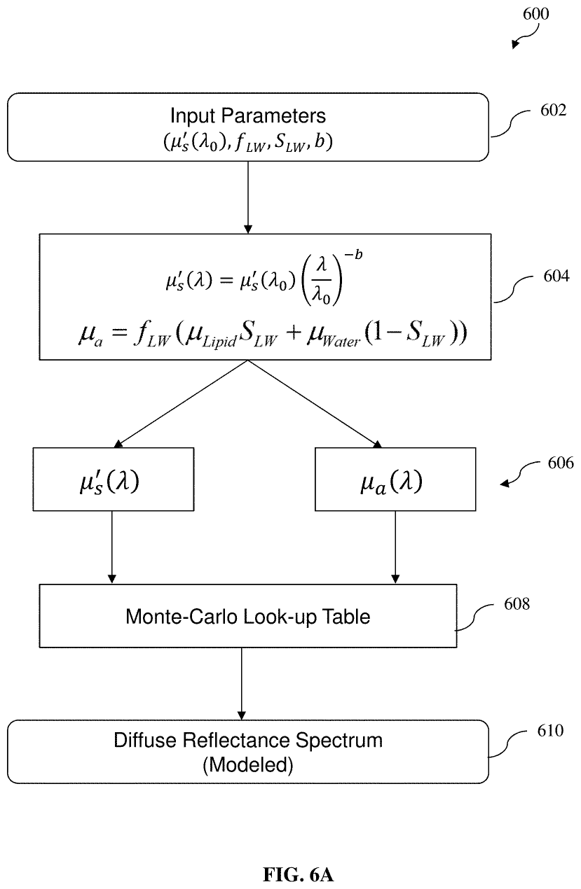

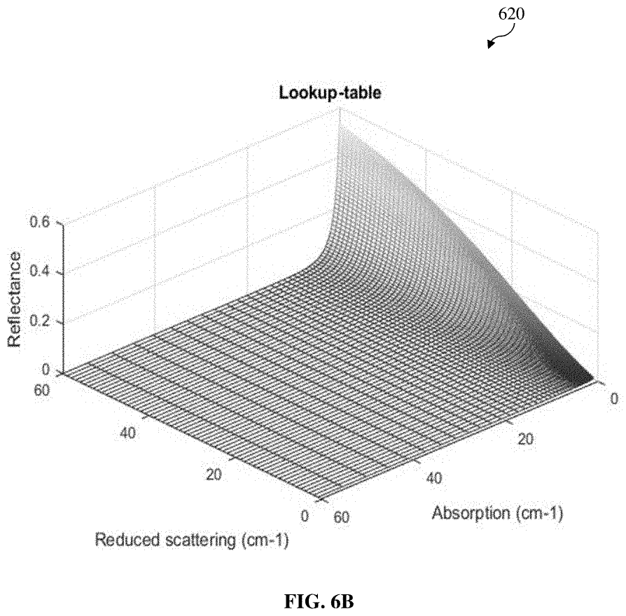

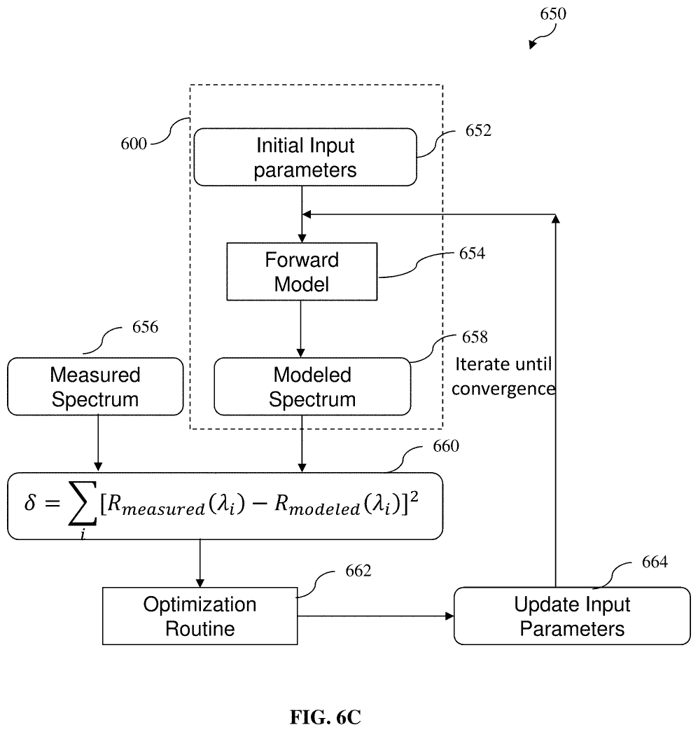

For a better understanding, FIG. 6A depicts a flow diagram of a method (forward model) 600 configured to generate a modeled diffuse reflectance spectrum using a MCLUT according to various example embodiments of the present invention, for the iteration process as described hereinbefore. At 602, various input parameters (e.g., the reduced scattering coefficient at reference wavelength (μ′ₛ(λ₀)), the scattering power coefficient (b), the volume fraction of lipid and water together (ƒ_{LW}) and the lipid fraction (S_{LW}) as described above in relation to Equations (2) to (4) above) for generating the modeled diffuse reflectance spectrum using the MCLUT are received. At 604 and 606, the reduced scattering coefficient μ′ₛ and absorption coefficient μₐ are computed (e.g., according to Equations (2) and (3) above). At 608 and 610, a modeled diffuse reflectance spectrum is generated using the MCLUT based on the reduced scattering coefficient μ′ₛ and absorption coefficient μₐ computed in 606. By way of an example illustration only and without limitation, an example MCLUT 620 for mapping various combinations of values of reduced scattering coefficient μ′ₛ (e.g., along an x-axis) and absorption coefficient μₐ (e.g., along a y-axis) to the corresponding diffuse reflectance value (e.g., along a z-axis) is shown in FIG. 6B.

For a better understanding, FIG. 6C depicts a flow diagram of a method (inverse model or iteration process) 650 configured to evaluate various input parameters (e.g., as described with respect to 602 of FIG. 6A) for minimizing the degree of difference (e.g., least square difference) between the modeled diffuse reflectance spectrum obtained and the measured diffuse reflectance spectrum obtained, so as to estimate various parameters, including the lipid fraction, at the adipose tissue site. In FIG. 6C, 652, 654 and 658 correspond to the method 600 of generating a modeled diffuse reflectance spectrum. At 660, a comparison between the diffuse reflectance spectrum information 656 obtained and the modeled diffuse reflectance spectrum 658 generated based on the input parameters at 652 is performed for determining a degree of similarity or difference between the diffuse reflectance spectrum information 656 obtained and the modeled diffuse reflectance spectrum 658 generated, as part of the iteration process for minimizing the degree of difference (e.g., least square difference) therebetween (with variable input parameters including the lipid fraction). As shown in FIG. 6C, the comparison may be configured as δ=Σᵢ[R_{measured}(λᵢ)−R_{modeled}(λᵢ)]², where δ denotes the sum of squares error and R denotes diffuse reflectance spectrum. At 662 and 664, based on the result of the comparison in 660, one or more input parameters may be varied or adjusted based on an optimization routine or process. The adjusted one or more input parameters may then be inputted to the forward model 654 for generating a modeled diffuse reflectance spectrum based on the input parameters, including the adjusted one or more input parameters as shown in FIG. 6C, and the iteration continues until a predetermined condition is met (e.g., convergence), such as the degree of difference (e.g., least square difference) between the diffuse reflectance spectrum information 656 obtained and the modeled diffuse reflectance spectrum 658 generated is considered to be optimal or minimum..

In various example embodiments, the input parameters which are adjusted or updated in the iteration process are only the volume fraction of lipid and water together (ƒ_{LW}) and the lipid fraction (S_{LW}), for estimating such parameters. In other words, only two variable parameters (ƒ_{LW} and S_{LW}) are iterated in the iteration process. It will be appreciated that the absorption coefficients of lipid and water are known values for a given range of wavelength. It will also be appreciated that, for example and without limitation, the method 650 (e.g., solving Equation 3 above) may be implemented using a MATLAB programming code.

Real-Time qPCR

In various example embodiments, total RNA was extracted with the RNeasy Lipid Tissue Mini Kit (Qiagen, Hilden, Germany) according to the manufacturer's instruction and Real-Time PCR was conducted using UCP1 primer pair UCP1_F: 5′-GGC CTC TAC GAC TCA GTC CA-3′ and UCP1_R: 5′-TAA GCC GGC TGA GAT CTT GT-3′. GAPDH was used to normalize the relative mRNA expression by using a primer pair GAPDH_F: 5′-CAA GGT CAT CCA CTT-3′ and GAPDH_R: 5′-GGC CAT CCA CAG TCT GG-3′. The results are represented as mean±SE (n=4 in each group).

### Western Blot Analysis

In various example embodiments, total protein lysates isolation from adipose tissues extracted from mice administered with either saline or CL (n=4, each group) was done in RadioImmunoPrecipitation Assay (RIPA) lysis buffer and quantified using the Biorad assay. Equal volume of proteins were loaded onto 10% Sodium Dodecyl Sulfate-Polyacrylamide Gel Electrophoresis (SDS-PAGE) followed by transfer to the PolyVinyliDene Fluoride (PVDF) membrane. Primary antibodies UCP1 (Abcam; 1:1000 dilution, Cambridge, UK) and loading control β-ACTIN (Santacruz Biotechnology; 1:2000, Dallas, Tex.) with secondary antibody goat anti-mouse-HRP (Santa Cruz Biotechnology; 1:5000) were used to detect horseradish peroxidase activity by chemiluminescent and autographed using X-ray film.

### Hematoxylin and Eosin (H&E) Staining and Immunohistochemistry (IHC)

In various example embodiments, H&E staining and UCP1 IHC were performed by the Advanced Molecular Pathology Laboratory (AMPL) at the Institute of Molecular and Cell Biology (IMCB), A*STAR, according to standard operating procedures. 1:50 dilution of UCP1 antibody (Abcam) at pH 6.0 was used for UCP1 IHC. All the images were obtained by using Olympus microscope equipped with DS-L3+Filc camera with a ×20 objective lens. Software ImageJ was used to analyse the images.

Various results and observations of the method of detecting adipose tissue browning as described above based on the example setup shown in FIG. 5 will now be described below.

### Estimation of Lipid Fraction as a Marker for Browning

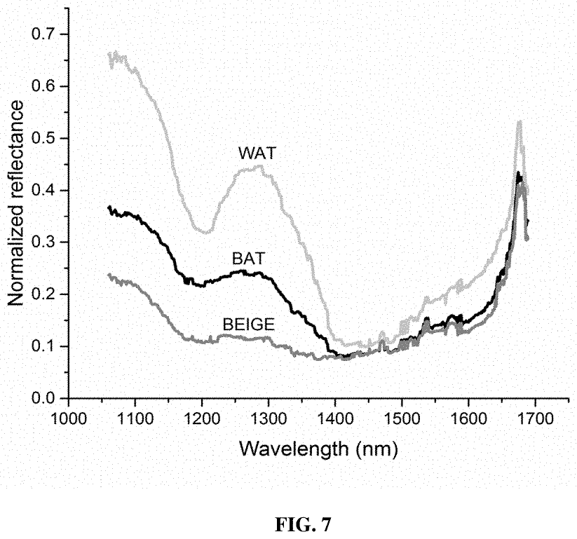

FIG. 7 depicts the typical or representative intensity normalized diffuse reflectance spectrum measured for BAT, WAT from the control mice and beige adipose tissue from the CL injected mice within 1050 to 1350 nm wavelength range. It clearly indicates that there is a marked difference in spectral profile among different adipose fat tissues in this spectral range. In various example embodiments, to quantitatively estimate the browning marker (the lipid fraction (S_{LW})), the background corrected reflectance spectrum is spectrally unmixed specifically for lipid and water chromophores using MCLUT.

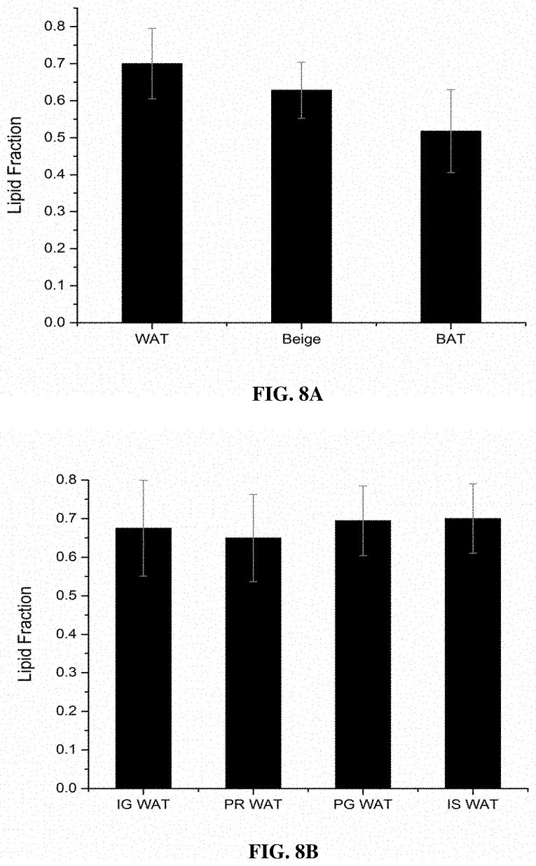

FIG. 8A shows the comparison of lipid fraction (S_{LW}) calculated for WAT, Beige adipose tissue and WAT in control mice. As shown in FIG. 8A, the lipid fraction evaluated for the beige adipose tissue (0.628±0.076 a.u.) was found to fall in between that of WAT (0.70±0.095 a.u.) and BAT (0.518±0.112 a.u.), that is, the measured S_{LW} is highest for WAT and lowest for BAT. This implies that the lipid content in the CL treated animals diminished over time during the browning and the calculated value S_{LW} indicates the WAT browning have occurred up to about 50% over a period of 7 days. This follows the established hypothesis and previous report in the art that activation of browning in WAT gives rise to enhanced lipolysis and fatty acid oxidation, which could result in the hydrolysis of stored triglycerides into free fatty acids and their catabolism. Further, there are established results from MRI studies that the lipid fraction values are significantly different between WAT and BAT in lean mice with BAT showing much lower value. However, MRI studies are not able to conclude the lipid fraction for beige and hence, DRS can be expanded as an optical modality to quantify beige by evaluating lipid fraction in NIR II optical window. Another advantage of calculating lipid fraction in NIR II optical is that the longer wavelengths can penetrate deep into the tissue and can be detected with customized fiber probe with large source-detector separations for future clinical translation. FIG. 8B shows the comparison of lipid fraction calculated for WAT in control mice at inguinal (IG WAT), perirenal (PR WAT) and perigonadal (PG WAT) and interscaplar (IS WAT). It is clearly evident from FIG. 8B that the lipid fraction for WAT from different depots have the similar value.

### Statistical Analysis of the DRS Data

The lipid fraction calculated from the measured diffuse reflectance spectrum for BAT, WAT and beige adipose tissue using MCLUT is statistically analysed using MINITAB statistical software. Each sample for BAT (sample size=8), WAT (sample size=15) and beige (sample size=14) is first statistically analysed using the Anderson-Darling normality test for t-test (sample normal distribution) and then 2-sample t-test is executed to identify difference of mean between two sample populations.

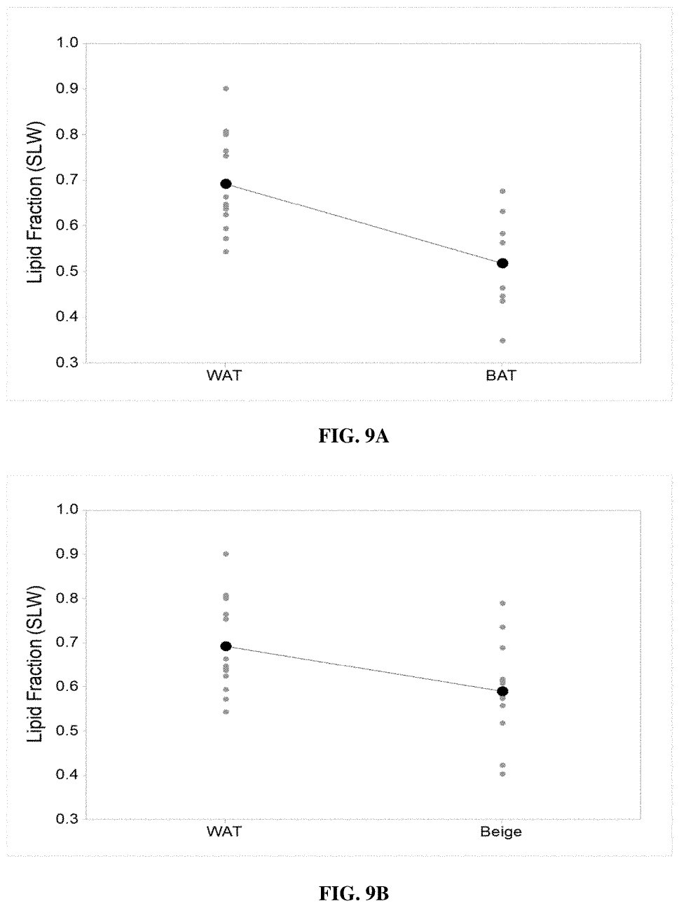

As the sample for BAT, WAT and beige adipose tissue satisfy for the normal distribution population, the mean for WAT population was statistically analyzed and differentiated from the BAT population using an independent 2-sample t-test. The independent 2-sample t-test helps to determine whether the two population means are different, that is, they are not related. FIGS. 9A and 9B show the individual plots, respectively, between WAT and BAT as well as WAT and beige to compare mean value of the sample. The independent 2-sample t-test between WAT and BAT gives the t-value=3.65 and p-value=0.003; whereas, the same test between WAT and beige adipose tissue derives t-value=2.66 and p-value=0.03. It is evident from these two independent t-tests that the resultant p-value is lower than the α-level 0.05, that is, the population mean is different in both the cases.

### Biochemical Analysis of Browning

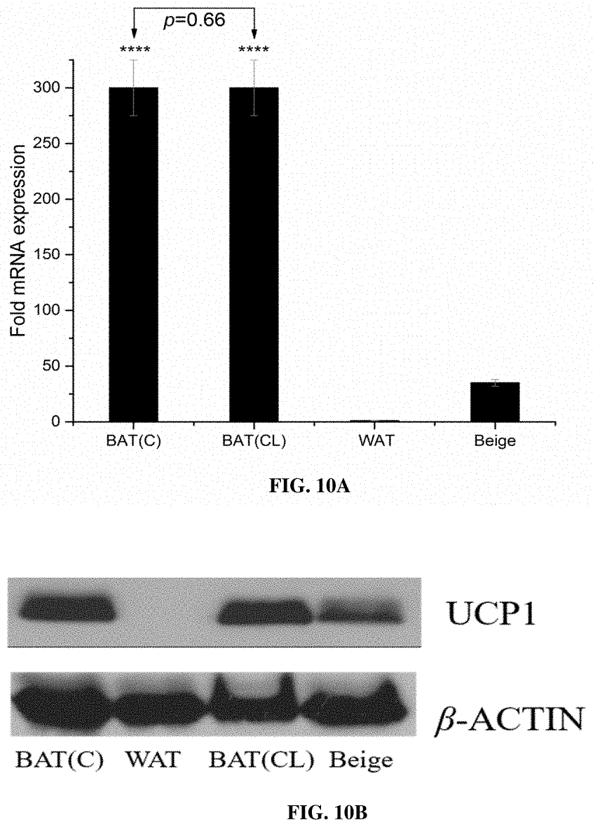

In order to determine if CL treatment leads to robust induction of WAT browning, gene and protein expression of BAT and beige-specific marker Ucp1 was analyzed in WAT, Beige and BAT tissues. Gene expression analysis by qPCR indicates that the transcript of Ucp1 is significantly enhanced upon CL treatment (beige) compared to the saline control (WAT) (FIG. 10A). Classical BAT showed higher Ucp1 expression than beige both under unstimulated and stimulated conditions. CL administration did not significantly change Ucp1 expression in BAT (FIG. 10A). The changes in UCP1 protein expression were also consistent with those in gene expression in that UCP1 protein was significantly induced by CL treatment in WAT albeit lower levels compared to control and stimulated BAT (FIG. 10B). Thus, these results suggest that CL treatment induced browning in WAT but not to the levels of classical BAT.

### H&E Staining and IHC as a Correlation for DRS Measurements

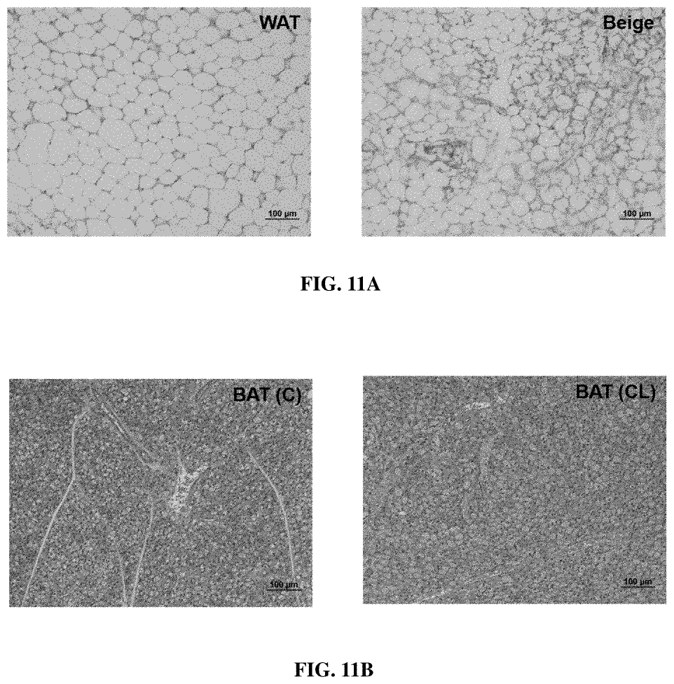

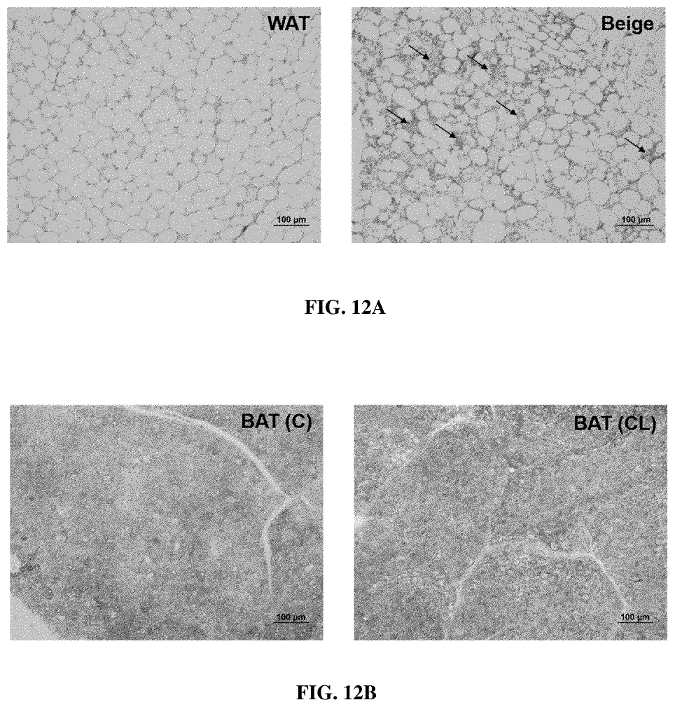

In addition, histological analyses of WAT, Beige and BAT were also performed to address the morphological changes of tissues induced by CL treatment. H&E staining shows that subpopulation of multilocular adipocytes are emerging in Beige tissue, in contrast to more homogeneous unilocular adipocytes in control WAT, indicating substantial browning of WAT by CL treatment (FIG. 11A). As expected, BAT contains predominantly multilocular brown adipocytes, whose appearance was not changed by CL treatment. The presence of beige and brown adipocytes was further confirmed by IHC with UCP1 protein. While control WAT showed faint UCP1 signals, CL treatment resulted in significant emergence of UCP1 expression in the areas of multilocular adipocytes in beige (see FIG. 12A). In contrast, UCP1 signals were found in the entire adipocyte population in BAT and not changed by CL treatment (see FIG. 12B). Together, these molecular and biochemical analyses confirmed robust browning into beige and its induction degrees in comparison to WAT and BAT.

Quantitative detection of browning of WAT is significant in understanding the mechanism of obesity and diabetes and developing a clinical strategy in managing metabolic disorders. Until now, common imaging approaches such as MRI and PET have been used to detect classical BAT and WAT in vivo in animals and humans. However, there has not been found to be any convincing existing results using these imaging modalities to detect the browning process of WAT in vivo, which is still an unmet need. This is primarily because only a certain subpopulation of white adipocytes in WAT can be induced to undergo browning compared to classical BAT. In contrast, various embodiments of the present invention provide a method or an approach of using DRS for the detection (e.g., quantitative detection) of browning process in vivo, which is advantageously fast (efficient), label-free, real-time, inexpensive and easy to implement. The DRS study in NIR II window according to various embodiments revealed that lipid fraction changes is consistent when tissues are undergoing browning. The DRS study advantageously found that lipid fraction values show a gradual decrease from WAT to BAT with beige adipose tissue exhibiting an intermediate value between that of BAT and WAT. The results obtained through various experiments were also confirmed with standard molecular and biochemical assays. The DRS study in NIR II window accordingly facilitates optical strategies to interrogate deeper into tissues compared to viable-NIR region. For example, interrogation of different layers in to fat depots in vivo may be realized according to various embodiments of the present invention by designing/configuring the DRS fiber probe with optimized excitation-collection fiber separation. Accordingly, various embodiments of the present invention advantageously provide and demonstrate the relative quantitative detection of browning process in vivo. Such a DRS approach advantageously facilitates the provision of simple and cost effective system for differentiating different fat depots and also for studying adipose browning that may open up new paradigm in managing various metabolic disorders.

While embodiments of the invention have been particularly shown and described with reference to specific embodiments, it should be understood by those skilled in the art that various changes in form and detail may be made therein without departing from the spirit and scope of the invention as defined by the appended claims. The scope of the invention is thus indicated by the appended claims and all changes which come within the meaning and range of equivalency of the claims are therefore intended to be embraced.

1. A system for in vivo detection of adipose tissue browning, the system comprising:
a fiber probe configured to illuminate light on an adipose tissue site;
a spectrometer configured to obtain diffuse reflectance spectrum information based on diffuse reflected light from the adipose tissue site in response to the light illuminated thereon;
a memory; and
at least one processor communicatively coupled to the memory and configured to:
determine a quantitative measure of a first type of chromophore at the adipose tissue site based on spectrally unmixing the diffuse reflectance spectrum information; and
detect adipose tissue browning at the adipose tissue site based on the quantitative measure of the first type of chromophore determined.

2. The system according to claim 1, wherein the diffuse reflectance spectrum information is spectrally unmixed based on a lookup table which, for each combination of a plurality of combinations of values of a plurality of tissue optical property parameters, maps the combination to a corresponding diffuse reflectance value.

3. The system according to claim 2, wherein the plurality of tissue optical property parameters comprises a reduced scattering coefficient and an absorption coefficient.

4. The system according to claim 3, wherein the absorption coefficient is dependent on the quantitative measure of the first type of chromophore at the adipose tissue site, and the quantitative measure of the first type of chromophore is determined based on a comparison between the diffuse reflectance spectrum information obtained and a modeled diffuse reflectance spectrum generated based on the quantitative measure of the first type of chromophore using the lookup table.

5. The system according to claim 4, wherein the absorption coefficient is dependent on quantitative measures of a plurality of types of chromophores, respectively, the plurality of types of chromophores including the first type of chromophore.

6. The system according to claim 5, wherein the quantitative measure of the first type of chromophore is a fraction of the first type of chromophore with respect to the plurality of types of chromophores.

7. The system according to claim 6, wherein the plurality of types of chromophores comprises lipid chromophore and water chromophore, and the first type of chromophore is the lipid chromophore.

8. The system according to claim 1, wherein the diffuse reflectance spectrum information is spectrally unmixed in a wavelength region of about 1050 nm to about 1400 nm.

9. The system according to claim 1, wherein the fiber probe comprises a source fiber channel and a plurality of detector fiber channels extending longitudinally within the fiber probe.

10. The system according to claim 9, wherein, in a cross-section of the fiber probe, the plurality of detector fiber channels has a circular arrangement about the source fiber channel.

11. A method of in vivo detection of adipose tissue browning, the method comprising:
illuminating light on an adipose tissue site using a fiber probe;
obtaining diffuse reflectance spectrum information based on diffuse reflected light from the adipose tissue site in response to the light illuminated thereon;
determining a quantitative measure of a first type of chromophore at the adipose tissue site based on spectrally unmixing the diffuse reflectance spectrum information; and
detecting adipose tissue browning at the adipose tissue site based on the quantitative measure of the first type of chromophore determined.

12. The method according to claim 11, wherein the diffuse reflectance spectrum information is spectrally unmixed based on a lookup table which, for each combination of a plurality of combinations of values of a plurality of tissue optical property parameters, maps the combination to a corresponding diffuse reflectance value.

13. The method according to claim 12, wherein the plurality of tissue optical property parameters comprises a reduced scattering coefficient and an absorption coefficient.

14. The method according to claim 13, wherein the absorption coefficient is dependent on the quantitative measure of the first type of chromophore at the adipose tissue site, and the quantitative measure of the first type of chromophore is determined based on a comparison between the diffuse reflectance spectrum information obtained and a modeled diffuse reflectance spectrum generated based on the quantitative measure of the first type of chromophore using the lookup table.

15. The method according to claim 14, wherein the absorption coefficient is dependent on quantitative measures of a plurality of types of chromophores, respectively, the plurality of types of chromophores including the first type of chromophore.

16. The method according to claim 15, wherein the quantitative measure of the first type of chromophore is a fraction of the first type of chromophore with respect to the plurality of types of chromophores.

17. The method according to claim 16, wherein the plurality of types of chromophores comprises lipid chromophore and water chromophore, and the first type of chromophore is the lipid chromophore.

18. The method according to claim 11, wherein the diffuse reflectance spectrum information is spectrally unmixed in a wavelength region of about 1050 nm to about 1400 nm.

19. The method according to claim 11, wherein the fiber probe comprises a source fiber channel and a plurality of detector fiber channels extending longitudinally within the fiber probe.

20. (canceled)

21. A method of detecting adipose tissue browning based on diffuse reflectance spectrum information, the method comprising:
receiving diffuse reflectance spectrum information with respect to an adipose tissue site;
determining a quantitative measure of a first type of chromophore at the adipose tissue site based on spectrally unmixing the diffuse reflectance spectrum information; and
detecting adipose tissue browning at the adipose tissue site based on the quantitative measure of the first type of chromophore determined.

22-28. (canceled)
